(19) United States
(12) Patent Application Publication
(10) Pub. No.: US 2026/0123918 A1
(43) Pub. Date: May 7, 2026

(54) QUANTITATIVE ULTRASOUND IMAGING METHOD AND APPARATUS USING LIGHTWEIGHT NEURAL NETWORK

(71) Applicant: BARRELEYE Inc, Daejeon (KR)

(72) Inventors: Hyeon-Min BAE, Daejeon (KR); Seokhwan OH, Daejeon (KR); Youngmin KIM, Daejeon (KR); Guil JUNG, Daejeon (KR); Hyeonjik LEE, Daejeon (KR); Myeong-Gee KIM, Daejeon (KR)

(21) Appl. No.: 19/114,548

(22) PCT Filed: Oct. 6, 2023
(86) PCT No.: PCT/KR2023/015455

§ 371 Date: Mar. 24, 2025

(30) Foreign Application Priority Data
Oct. 31, 2022 (KR) 10-2022-0142583
Oct. 5, 2023 (KR) 10-2023-0132844

(51) Int. Cl. A61B 8/00; G06T 7/62

(52) U.S. Cl. CPC A61B 8/5223; A61B 8/4427; A61B 8/5207; G06T 7/62; G06T 2207/10132; G06T 2207/20081; G06T 2207/20084; G06T 2207/30004

(57) ABSTRACT

An operating method of an imaging apparatus operated by at least one processor includes receiving ultrasound data of a tissue and generating a quantitative image representing a distribution of quantitative variables in the tissue from the ultrasound data using a lightweight neural network trained upon receiving knowledge of a teacher neural network.

### TECHNICAL FIELD

The disclosure relates to ultrasound imaging.

### BACKGROUND ART

Cancer is challenging to detect in its early stages, necessitating periodic diagnosis and continuous monitoring of lesion size and characteristics. Common imaging modalities for this purpose include X-ray, magnetic resonance imaging (MRI), computed tomography (CT), and ultrasound. While X-ray, MRI, and CT have the disadvantages such as radiation exposure risk, long scan times, and high costs, the ultrasound imaging is safe, relatively inexpensive, and capable of providing real-time images, allowing users to monitor lesions in real time and obtain desired images.

Currently, the most commercialized ultrasound imaging equipment is a brightness mode (B-mode) imaging system. B-mode imaging identifies the location and size of an object through measuring the time and the strength for which ultrasound waves are reflected and returned from a surface of the object. Since this method finds a location of the lesion in real time, users may efficiently obtain desired images while monitoring lesions in real time, and this method is safe, relatively inexpensive, and has good accessibility. However, there are disadvantages in that image quality is not maintained consistently depending on the user's skill level, and quantitative characteristics cannot be imaged. In other words, since the B-mode technique provides only structural information of tissues, the sensitivity and specificity may be low in a differential diagnosis of benign and malignant tumors distinguished by histological characteristics.

Recently, research has been conducted to reconstruct biomechanical characteristics in real time through quantitative ultrasound imaging. However, since a neural network performing quantitative ultrasound imaging requires extensive parallel calculation, it is not easy to apply the neural network to existing ultrasound imaging devices. This challenge is particularly pronounced in mobile ultrasound imaging devices, which has limited computational resources, restricting the feasibility of applying quantitative ultrasound imaging.

### DISCLOSURE

### Technical Problem

The disclosure attempts to provide a quantitative ultrasound imaging method and apparatus using a lightweight neural network.

The disclosure also attempts to provide a lightweight neural network through knowledge distillation and/or neural network parameter quantization.

### Technical Solution

According to an exemplary embodiment, an operating method of an imaging apparatus operated by at least one processor includes: receiving ultrasound data of a tissue and generating a quantitative image representing a distribution of quantitative variables in the tissue from the ultrasound data using a lightweight neural network trained upon receiving knowledge of a teacher neural network.

The lightweight neural network may be configured to extract quantitative features from the ultrasound data using multi-stage separable convolution and to reconstruct the quantitative features to output the quantitative image.

The lightweight neural network may become lightweight through neural network parameter quantization and be configured to extract quantitative features from the ultrasound data and to reconstruct the quantitative features to output the quantitative image.

The lightweight neural network may be an artificial intelligence model trained using knowledge for feature map extraction and knowledge for quantitative image restoration, which are received from the teacher neural network.

The lightweight neural network may be an artificial intelligence model trained by using an objective function including a first loss related to a difference from a correct image, a second loss related to a difference from a feature map extracted from the teacher neural network, and a third loss related to a difference from the quantitative image generated from the teacher neural network.

The quantitative variables may include at least one of attenuation coefficient (AC), speed of sound (SoS), effective scatterer concentration (ESC), and effective scatterer diameter (ESD).

The imaging apparatus may be a mobile device.

According to an exemplary embodiment, an imaging apparatus includes: a memory; and a processor configured to execute instructions stored in the memory, wherein the processor is configured to generate a quantitative image representing a distribution of quantitative variables in a tissue from ultrasound data of the tissue using a lightweight neural network trained upon receiving knowledge from a teacher neural network.

The lightweight neural network may be configured to extract quantitative features from the ultrasound data using multi-stage separable convolution and to reconstruct the quantitative features to output the quantitative image.

The lightweight neural network may become lightweight through neural network parameter quantization and be configured to extract quantitative features from the ultrasound data and to reconstruct the quantitative features to output the quantitative image.

The lightweight neural network may be an artificial intelligence model trained upon receiving knowledge for feature map extraction and knowledge for quantitative image restoration from the teacher neural network.

The lightweight neural network may be an artificial intelligence model trained using an objective function including a first loss related to a difference from a correct image, a second loss related to a difference from a feature map extracted from the teacher neural network, and a third loss related to a difference from the quantitative image generated from the teacher neural network.

The quantitative variables may include at least one of attenuation coefficient (AC), speed of sound (SoS), effective scatterer concentration (ESC), and effective scatterer diameter (ESD).

The imaging apparatus may be a mobile device.

According to an exemplary embodiment, a computer program includes instructions stored in a computer-readable storage medium and executed by a processor, wherein the computer program includes instructions executing an encoder configured to receive ultrasound data of a tissue and extract a quantitative feature map from the ultrasound data, and a decoder configured to reconstruct a quantitative image representing a distribution of quantitative parameters in the tissue from the quantitative feature map, wherein the encoder and the decoder are lightweight neural networks trained using feature map extraction knowledge and image reconstruction knowledge transmitted from a teacher neural network.

The encoder may be a model configured to extract quantitative features from the ultrasound data using multi-stage separable convolution.

The encoder and the decoder may become lightweight through neural network parameter quantization and be configured to extract quantitative features from the ultrasound data and reconstruct the quantitative features to output the quantitative image.

The encoder and the decoder may be artificial intelligence model trained using an objective function including a first loss related to a difference from a correct image, a second loss related to a difference from a feature map extracted from the teacher neural network, and a third loss related to a difference from the quantitative image generated from the teacher neural network.

The quantitative variables may include at least one of attenuation coefficient (AC), speed of sound (SoS), effective scatterer concentration (ESC), and effective scatterer diameter (ESD).

### Advantageous Effects

According to an exemplary embodiment, since a high-quality quantitative image may be reconstructed in real time through a lightweight neural network, it is possible to provide high-quality quantitative images even in an ultrasound device with limited resources, such as a mobile ultrasound apparatus.

According to an exemplary embodiment, the reconstruction accuracy of a lightweight neural network may be improved through knowledge distillation.

According to an exemplary embodiment, the number of parameters of a lightweight neural network may be reduced by 96% or more compared to the existing neural network, thereby significantly lowering computing resources required for ultrasound quantitative imaging.

According to an exemplary embodiment, reducing computing resources required for quantitative ultrasound imaging can be lower product costs and expand the utilization of various types of ultrasound devices, including mobile ultrasound devices.

### DESCRIPTION OF THE DRAWINGS

FIG. 1 is a conceptual diagram illustrating a quantitative ultrasound imaging apparatus according to an exemplary embodiment.

FIG. 2 is a diagram illustrating a neural network according to an exemplary embodiment.

FIG. 3 is a diagram illustrating a multi-stage separable convolution.

FIG. 4 is a diagram illustrating neural network training according to an exemplary embodiment.

FIG. 5 is a flowchart of a quantitative ultrasound imaging method according to an exemplary embodiment.

FIG. 6 is a hardware configuration diagram of an imaging apparatus according to an exemplary embodiment.

### MODE FOR INVENTION

In the following detailed description, only certain exemplary embodiments of the disclosure have been shown and described, simply by way of illustration. As those skilled in the art would realize, the described exemplary embodiments may be modified in various different ways, all without departing from the spirit or scope of the disclosure. Accordingly, the drawings and description are to be regarded as illustrative in nature and not restrictive. Like reference numerals designate like elements throughout the specification.

Throughout the specification, unless explicitly described to the contrary, the word “comprise”, and variations, such as “comprises” or “comprising”, will be understood to imply the inclusion of stated elements but not the exclusion of any other elements. In addition, the terms “-er”, “-or”, and “module” described in the specification mean units for processing at least one function and operation, and may be implemented by hardware components or software components, and combinations thereof.

The neural network of the disclosure is an artificial intelligence model that learns at least one task and may be implemented as a software/program that runs on a computing device. The program is stored in a non-transitory storage medium and includes instructions that are described to cause a processor to execute the operation of the disclosure. The program may be downloaded through a network or sold in the form of a product.

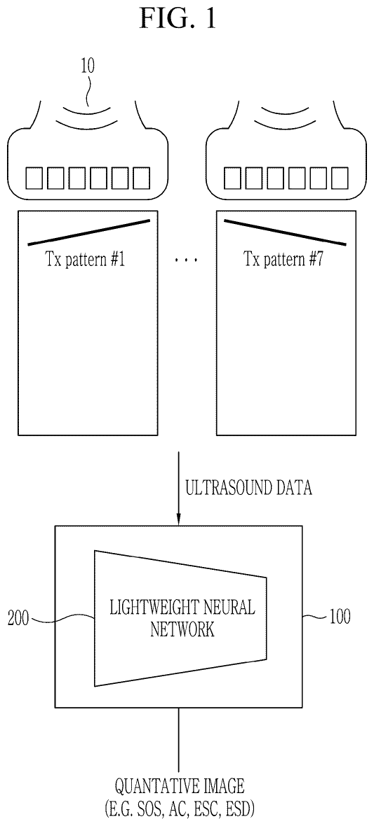

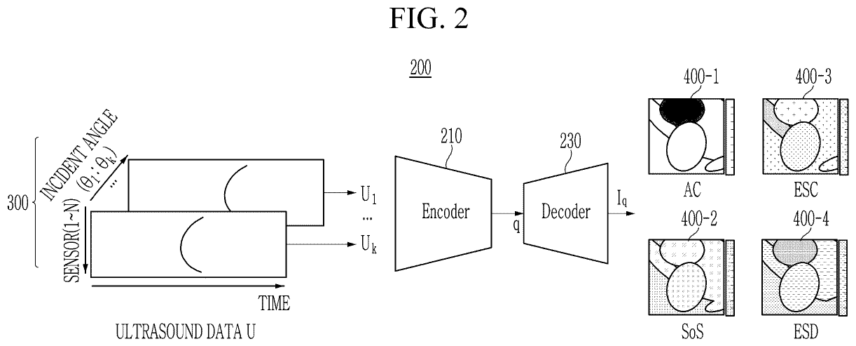

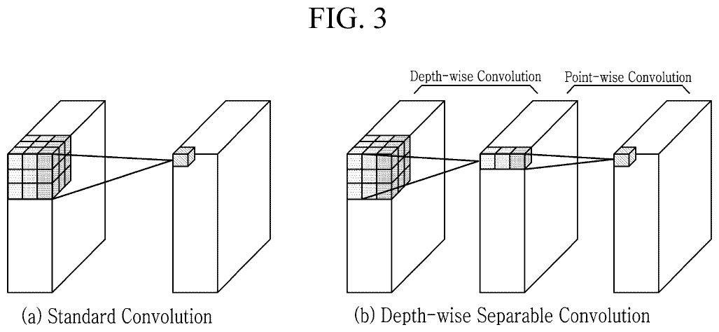

FIG. 1 is a conceptual diagram illustrating a quantitative ultrasound imaging apparatus according to an exemplary embodiment, FIG. 2 is a diagram illustrating a neural network according to an exemplary embodiment, and FIG. 3 is a diagram illustrating a multi-stage separable convolution.

Referring to FIG. 1, a quantitative ultrasound imaging apparatus (simply referred to as an “imaging apparatus”) 100 is a computing apparatus operated by at least one processor, which receives ultrasound data obtained from a tissue through an ultrasound probe 10. The imaging apparatus 100 includes a memory that stores instructions and a processor that executes the instructions, and the processor executes the instructions included in the computer program to perform the operation of the disclosure.

The imaging apparatus 100 may be implemented to interwork with a plurality of ultrasound devices through a communication network or may be integrated into an ultrasound device to be implemented. The imaging apparatus 100 may be implemented in a device with limited available computing resources (e.g., memory, processor, etc.) to provide quantitative images and may be implemented in, for example, various types of mobile devices.

The imaging apparatus 100 may generate a quantitative image for at least one variable representing the characteristics of a tissue by using a neural network 200 that extracts quantitative characteristics of the tissue from ultrasound data. Here, the imaging apparatus 100 may output images for quantitative variables of the tissue, such as an attenuation coefficient (AC), a speed of sound (SoS), an effective scatterer concentration (ESC) representing a density distribution within the tissue, and an effective scatterer diameter (ESD) representing the size of cells within the tissue.

The neural network 200 is an artificial intelligence model capable of learning at least one task and may be implemented as software/program running on a computing device. The neural network 200 is a lightweight neural network and may be referred to as mobile friendly quantitative ultrasound imaging network (MQI-Net) in that it may be applied to devices with limited available resources, such as mobile devices.

Meanwhile, the attenuation coefficient (AC), the speed of sound (SoS), the effective scatterer concentration (ESC), and the effective scatterer diameter (ESD) are variables known as biomarkers for lesion extraction and are closely related to biomechanical characteristics of the tissue. Therefore, as more variables are used, comprehensive analysis of the lesion may be performed, which may increase the diagnostic sensitivity and specificity.

Ultrasound data used to generate a quantitative image of the tissue may be obtained from the ultrasound probe 10. The ultrasound probe 10 is a probe that may emit ultrasound signals and obtain ultrasound data reflected from the tissue. The ultrasound signal radiated to the tissue may be a plane wave. The ultrasound probe 10 may include N (e.g., 128) ultrasound sensors arranged therein and may vary in type depending on the arrangement shape. The sensors may be implemented as piezoelectric elements. In addition, the ultrasound probe 10 may be a phased array probe that generates ultrasound signals by applying electrical signals to each piezoelectric element at regular time intervals.

The ultrasound probe 10 may irradiate ultrasound signals of different beam patterns (Tx pattern #1 to #k) onto the tissue and obtain radio frequency (RF) data reflected and returned from the tissue. The ultrasound data is RF data obtained using plane waves having k different incident angles, and for example, the incident angles may be set to −15°, −10°, −5°, 0°, 5°, 10°, and 15°. Here, the ultrasound data may include not only RF data obtained from the ultrasound probe 10 but also data synthesized from the obtained RF data.

Meanwhile, the ultrasound data obtained from the ultrasound probe 10 includes information on a delay time for receiving the reflected ultrasound signal for each sensor of the ultrasound probe 10. Therefore, the ultrasound data may be expressed as an image representing the delay time information for each sensor.

Referring to FIG. 2, the lightweight neural network 200 may include an encoder 210 and a decoder 230 that extract a quantitative feature q from ultrasound data U (U1˜Uk) 300 obtained from the tissue and reconstruct a quantitative image I_{q} from the quantitative feature. The structures of the encoder 210 and the decoder 230 may be designed variously.

The lightweight neural network 200 may generate the quantitative image I_{q} representing a distribution of quantitative variables in the tissue from the ultrasound data. The neural network 200 may generate, for example, an attenuation coefficient image 400-1, a sound velocity image 400-2, a scatterer density image 400-3, a scatterer size image 400-4, etc., and may generate quantitative images for a plurality of quantitative variables.

The neural network structure may be designed variously. For example, the lightweight neural network 200 may perform conditional encoding that variably extracts quantitative features according to a target variable to be reconstructed from the ultrasound data, and through this, may generate multi-parameter quantitative images of the tissue in a complex manner. Here, the conditional encoding conditionally changes network parameters of an encoding path according to a selected variable and may improve image restoration performance for the corresponding variable, so that a quantitative feature of the variable to be reconstructed from the ultrasound data is optimally extracted. The encoder and decoder structures for generating a quantitative image may be designed variously.

For example, the encoder 210 may compress an ultrasound data image U˜R^{128×3018×7} into the feature map q˜R^{16×16×512}. The ultrasound data image may be expressed by the number of probe sensors (transducer elements) (N=128), time axis indices (e.g., t=3018), and beam patterns (k=7). The encoder 210 may configure an encoding network with various network models/network blocks, and the encoding path may be configured to sequentially perform convolution operations, activations (e.g., ReLU), and down-sampling.

The decoder 230 may receive a feature map q output from the encoder 210 and reconstruct the quantitative image I_{q} from the feature map q. For example, the decoder 230 may generate a quantitative image I_{q}˜R^{128×128} from the feature map q˜R^{16×16×512}. The decoder 230 may configure a decoding network with various network models/network blocks. For example, the decoder 230 may generate a high-resolution quantitative image I_{q} by an up-sampling method. Alternatively, the decoder 230 may generate a high-resolution quantitative image I_{q} by using parallel multi-resolution subnetworks based on a high-resolution network (HRNet). The decoder 230 including parallel multi-resolution subnetworks may sequentially perform multi-resolution convolution from a low-resolution subnetwork to ultimately generate a high-resolution quantitative image I_{q}, while increasing the image resolution. An output layer of the decoder 230 may merge to the highest resolution representation and generate the high-resolution quantitative image I_{q} synthesized through 1×1 convolution.

The lightweight neural network 200 may encode features using multi-stage separable convolution. The multi-stage separable convolution may vary, and for example, may include depth-wise separable convolution.

Referring to (a) of FIG. 3, a general convolution filter performs spatial and channel-wise operations simultaneously. In contrast, referring to (b) of FIG. 3, the multi-stage separable convolution separates them and sequentially performs depth-wise convolution and point-wise convolution.

The depth-wise convolution convolves the same kernel for each channel of an input. Point-wise convolution combines output channels of depth-wise convolution by applying a 1×1 kernel convolution filter. By using this multi-stage separable convolution, the lightweight neural network 200 may be made lightweight by reducing the general processing redundancy of the general convolution method and may improve efficiency.

Meanwhile, the lightweight neural network 200 may be made lightweight through neural network parameter quantization. Quantization may be performed by expressing parameters of weights of a neural network and activation function as integers or a small number of bits or by reducing the precision to lower computational complexity. Neural network parameter quantization may be performed during a training process of the lightweight neural network or after training is completed.

In this manner, the lightweight neural network 200 may reduce a model size, increase a calculation speed, and reduce a memory usage by reducing the number of parameters by 96% compared to the neural network using the general convolution method. In general, when the number of parameters of the neural network is reduced, a reconstruction accuracy may decrease, but the neural network 200 may improve the performance through knowledge distillation. The lightweight neural network 200 may be made lightweight while increasing the accuracy through techniques, such as multi-stage separable convolution, neural network parameter quantization, and knowledge distillation.

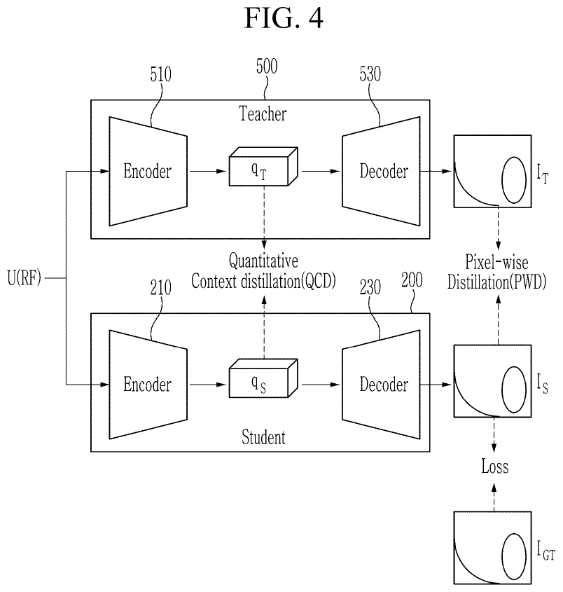

FIG. 4 is a diagram illustrating neural network training according to an exemplary embodiment.

Referring to FIG. 4, the lightweight neural network 200 may be trained based on knowledge distillation. Knowledge distillation is a method of training a student neural network by transferring the knowledge of a teacher neural network to the student neural network. The lightweight neural network 200 may be trained by a separate computing device.

The lightweight neural network 200 may be a student neural network that learns by receiving knowledge from the teacher neural network 500 and may be a lightweight model with fewer parameters than the teacher neural network 500. The teacher neural network 500 is a model configured to extract a feature map q_{T} from ultrasound data U (RF) and reconstruct a quantitative image I_{T} from the feature map q_{T} and may be a large-scale artificial intelligence model that uses a large number of parameters. The teacher neural network 500 may be designed with an encoder 510 and a decoder 530 structure.

The lightweight neural network 200 may include an encoder 210 that receives ultrasound data of a tissue and extracts a quantitative feature map from the ultrasound data and a decoder 230 that reconstructs a quantitative image representing a distribution of quantitative variables in the tissue from the quantitative feature map. The encoder 210 may be a lightweight model trained by using feature map extraction knowledge transferred from the teacher neural network 500, and the decoder 230 may be a lightweight model trained by using image restoration knowledge transferred from the teacher neural network.

The lightweight neural network 200 is configured to extract a feature map q_{S} from the ultrasound data U (RF) and reconstruct the quantitative image I_{S} from the feature map q_{S} and may be trained by using the knowledge transferred from the teacher neural network 500, as well as restoration/reconstruction loss with a correct image (Ground truth) I_{GT} during the training process. The lightweight neural network 200 may receive the knowledge for feature map extraction and the knowledge for quantitative image restoration from the teacher neural network 500. The knowledge for feature map extraction may be referred to as quantitative context distillation (QCD) knowledge, and the knowledge for quantitative image restoration may be referred to as pixel-wise distillation (PWD) knowledge.

The quantitative context distillation (QCD) knowledge serves to transfer the feature map extraction method of the teacher neural network 500 to the lightweight neural network 200, and the encoder 210 is trained to encode the feature map q_{S} of the ultrasound data U (RF) close to the feature map q_{T} of the teacher neural network 500 by the quantitative context distillation (QCD) knowledge.

The pixel-wise distillation (PWD) knowledge serves to transfer the image restoration method of the teacher neural network 500 to the lightweight neural network 200, and the decoder 230 is trained to reconstruct the quantitative image I_{S} close to the quantitative image I_{T} output from the teacher neural network 500 by the pixel-wise distillation (PWD) knowledge.

An objective function θ* used for training the lightweight neural network 200 may be defined as a loss function as in Equation 1, and the lightweight neural network 200 may be trained to minimize the loss function. The lightweight neural network 200 is trained to minimize the loss L_{MSE} related to a difference between the generated quantitative image I_{S} and the correct image I_{GT}, and here, the lightweight neural network 200 is trained by reflecting L_{QCD} and L_{PWD}, which are losses related to the difference from the knowledge of the teacher neural network 500. The degree of knowledge transfer from the teacher neural network 500 may be controlled by hyperparameters β and λ. L_{MSE}, L_{QCD}, and L_{PWD} of Equation 1 may be defined as in Equations 2, 3, and 4, but are not limited thereto.

$${θ}^{*} = arg min E [ LMSE + β LQCD + λ LPWD ] \qquad ( Equation 1 )$$

$${L}_{M S E} = {\sum }_{i = 1}^{n} {( {I}_{S} - {I}_{G T} )}^{2} \qquad ( Equation 2 )$$

$${L}_{Q C D} = \frac{{\sum }_{i = 1}^{n} ( {q}_{T} - {q}_{S} )}{C \times H \times W} \qquad ( Equation 3 )$$

$${L}_{P W D} = \frac{{\sum }_{i = 1}^{n} ( {I}_{T} - {I}_{S} )}{H \times W} \qquad ( Equation 4 )$$

In Equation 1, L_{MSE} is a loss related to the difference between the quantitative image I_{S} output from the lightweight neural network 200 and the correct image I_{GT} and may be calculated as a mean squared error (MSE) as in Equation 2.

In Equation 1, L_{QCD} is a loss related to a difference between the feature map q_{S} extracted from the lightweight neural network 200 and the feature map q_{T} extracted from the teacher neural network 500 and may be expressed as a quantitative context distillation (QCD) loss as in Equation 3. In Equation 3, C, H, and W are the channel, height, and width of the feature map, respectively. By L_{QCD}, the encoder 210 may be trained to minimize an L1 norm of the feature map q_{S} and the feature map q_{T}.

In Equation 1, L_{PWD} is a loss related to a difference between the quantitative image I_{S} generated by the lightweight neural network 200 and the quantitative image I_{T} generated by the teacher neural network 500 and may be expressed as a pixel-wise distillation (PWD) loss as in Equation 4. In Equation 4, H and W are the height and width of the image, respectively. The decoder 230 may be trained to minimize the difference between the quantitative image I_{S} and the quantitative image I_{T} by L_{PWD}.

Learning data of the lightweight neural network 200 may include ultrasound data obtained from various human environments and may be collected using an ultrasound simulation tool. For simulation phantoms representing organs and lesions, a speed of sound (SoS) distribution, attenuation coefficient (AC) distribution, and a density distribution may be modeled so that a human body is imitated, while being simple and not losing generality.

For example, in the simulation, a region of Interest (RoI) is set to 45 mm×45 mm, and ellipses with radii of 2 to 30 mm may be randomly disposed. A background and ellipses may have a speed of sound (SoS) in the range of 1400 to 1700 m/s, an attenuation coefficient in the range of 0 to 1.5 dB/cm/MHz, and a density value in the range of 0.85 to 1.15 kg/m³. Speckles having a size of 0 to 150 μm may be distributed to represent scatterer density and scatterer size.

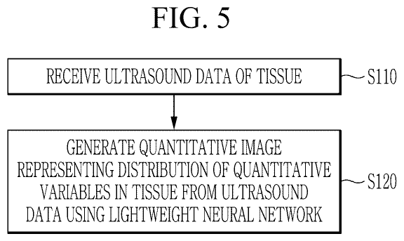

FIG. 5 is a flowchart of a quantitative ultrasound imaging method according to an exemplary embodiment.

Referring to FIG. 5, the imaging apparatus 100 receives ultrasound data of a tissue (S110). The ultrasound data may be RF data obtained by using plane waves having k different incident angles.

The imaging apparatus 100 generates a quantitative image representing a distribution of quantitative variables in the tissue from ultrasound data using the lightweight neural network 200 (S120). The lightweight neural network 200 is configured to extract a quantitative feature from the ultrasound data using a multi-stage separable convolution, thereby reducing the general processing duplication of a general convolution method and thereby reducing weight and improving efficiency. The lightweight neural network 200 may be reduced in weight through neural network parameter quantization. The lightweight neural network 200 is trained upon receiving a feature map extraction method of the teacher neural network 500 through quantitative context distillation (QCD) knowledge and receiving an image restoration method of the teacher neural network 500 through pixel-wise distillation (PWD) knowledge, thereby increasing reconstruction accuracy and improving performance.

The lightweight neural network 200 may be trained to reconstruct quantitative images of an attenuation coefficient (AC), speed of sound (SoS), an effective scatterer concentration (ESC), an effective scatterer diameter (ESD), etc. from the ultrasound data. The structure of the lightweight neural network 200 may be designed variously. For example, conditional encoding may be performed to variably extract quantitative features according to a target variable to be reconstructed from the ultrasound data, and a quantitative image may be reconstructed from the extracted quantitative feature map.

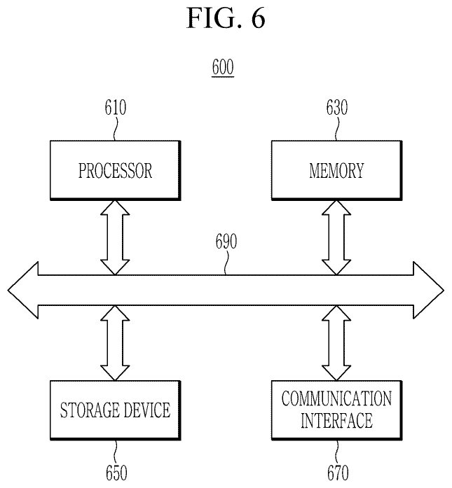

FIG. 6 is a hardware configuration diagram of an imaging apparatus according to an exemplary embodiment.

Referring to FIG. 6, the imaging apparatus 100 may be a computing device 600 operated by at least one processor and connected to the ultrasound probe 10 or a device that provides data obtained from the ultrasound probe 10.

The computing device 600 may include one or more processors 610, a memory 630 that loads a program executed by the processor 610, a storage 650 that stores the program and various data, a communication interface 670, and a bus 690 that connects these components. In addition, the computing device 600 may further include various components. The program may include instructions that cause the processor 610 to perform methods/operations according to various exemplary embodiments of the disclosure when loaded into the memory 630. That is, the processor 610 may perform methods/operations according to various exemplary embodiments of the disclosure by executing the instructions. The instructions are a series of computer-readable instructions grouped based on functions and are components of a computer program and are executed by the processor.

The processor 610 controls the overall operation of each component of the computing device 600. The processor 610 may be configured to include at least one of a central processing unit (CPU), a micro processing unit (MPU), a micro controller unit (MCU), a graphics processing unit (GPU), or any other type of processor well known in the art of the disclosure. In addition, the processor 610 may perform arithmetic operations for at least one application or program for executing methods/operations according to various exemplary embodiments of the disclosure.

The memory 630 stores various data, instructions, and/or information. The memory 630 may load one or more programs from the storage 650 to execute the methods/operations according to various exemplary embodiments of the disclosure. The memory 630 may be implemented as a volatile memory, such as RAM, but the scope of the disclosure is not limited thereto.

The storage 650 may non-temporarily store the program. The storage 650 may be configured to include a non-volatile memory, such as a read-only memory (ROM), an erasable programmable ROM (EPROM), an electrically erasable programmable ROM (EEPROM), a flash memory, a hard disk, a removable disk, or any form of a computer-readable recording medium well known in the art to which the disclosure pertains.

The communication interface 670 supports wired and wireless communication of the computing device 600. To this end, the communication interface 670 may be configured to include a communication module well known in the art of the disclosure.

The bus 690 provides a communication function between components of the computing device 600. The bus 690 may be implemented as various types of buses, such as an address bus, a data bus, and a control bus.

In this manner, according to an exemplary embodiment, since a high-quality quantitative image may be reconstructed in real time through the lightweight neural network, a high-quality quantitative image may be provided even in an ultrasound device with limited resources, such as a mobile ultrasound device.

According to an exemplary embodiment, the reconstruction accuracy of the lightweight neural network may be improved through knowledge distillation.

According to an exemplary embodiment, the number of parameters of a lightweight neural network may be reduced by 96% or more compared to the existing neural network, thereby significantly lowering computing resources required for ultrasound quantitative imaging.

According to an exemplary embodiment, reducing computing resources required for quantitative ultrasound imaging can be lower product costs and expand the utilization of various types of ultrasound devices, including mobile ultrasound devices.

The exemplary embodiments of the disclosure may not necessarily be implemented only through the foregoing devices and methods but may also be implemented through a program for realizing functions corresponding to the configurations of the exemplary embodiments of the disclosure, a recording medium including the program, or the like.

The exemplary embodiments of the disclosure have been described in detail, but the scope of the disclosure is not limited thereto and various variants and modifications by a person skilled in the art using a basic concept of the disclosure defined in claims also belong to the scope of the disclosure.

1. An operating method of an imaging apparatus operated by at least one processor, the operating method comprising:
receiving ultrasound data of a tissue and
generating a quantitative image representing a distribution of quantitative variables in the tissue from the ultrasound data using a lightweight neural network trained upon receiving knowledge of a teacher neural network.

2. The operating method of claim 1, wherein the lightweight neural network is configured to
extract quantitative features from the ultrasound data using multi-stage separable convolution and reconstruct the quantitative features to output the quantitative image.

3. The operating method of claim 1, wherein the lightweight neural network becomes lightweight through neural network parameter quantization and is configured to
extract quantitative features from the ultrasound data and reconstruct the quantitative features to output the quantitative image.

4. The operating method of claim 1, wherein the lightweight neural network is an artificial intelligence model trained using knowledge for feature map extraction and knowledge for quantitative image restoration, which are received from the teacher neural network.

5. The operating method of claim 4, wherein the lightweight neural network is an artificial intelligence model trained by using an objective function including a first loss related to a difference from a correct image, a second loss related to a difference from a feature map extracted from the teacher neural network, and a third loss related to a difference from the quantitative image generated from the teacher neural network.

6. The operating method of claim 1, wherein the quantitative variables include
at least one of attenuation coefficient (AC), speed of sound (SoS), effective scatterer concentration (ESC), and effective scatterer diameter (ESD).

7. The operating method of claim 1, wherein the imaging apparatus is a mobile device.

8. An imaging apparatus comprising:
a memory; and
a processor configured to execute instructions stored in the memory,
wherein the processor is configured to
generate a quantitative image representing a distribution of quantitative variables in a tissue from ultrasound data of the tissue using a lightweight neural network trained upon receiving knowledge from a teacher neural network.

9. The imaging apparatus of claim 8, wherein the lightweight neural network is configured to
extract quantitative features from the ultrasound data using multi-stage separable convolution and reconstruct the quantitative features to output the quantitative image.

10. The imaging apparatus of claim 8, wherein the lightweight neural network becomes lightweight through neural network parameter quantization and is configured to extract quantitative features from the ultrasound data and reconstruct the quantitative features to output the quantitative image.

11. The imaging apparatus of claim 8, wherein the lightweight neural network is an artificial intelligence model trained upon receiving knowledge for feature map extraction and knowledge for quantitative image restoration from the teacher neural network.

12. The imaging apparatus of claim 11, wherein the lightweight neural network is an artificial intelligence model trained using an objective function including a first loss related to a difference from a correct image, a second loss related to a difference from a feature map extracted from the teacher neural network, and a third loss related to a difference from the quantitative image generated from the teacher neural network.

13. The imaging apparatus of claim 8, wherein the quantitative variables include
at least one of attenuation coefficient (AC), speed of sound (SoS), effective scatterer concentration (ESC), and effective scatterer diameter (ESD).

14. The imaging apparatus of claim 8, wherein the imaging apparatus is a mobile device.

15. A computer program including instructions stored in a computer-readable storage medium and executed by a processor, wherein the computer program includes instructions executing
an encoder configured to receive ultrasound data of a tissue and extract a quantitative feature map from the ultrasound data, and
a decoder configured to reconstruct a quantitative image representing a distribution of quantitative variables in the tissue from the quantitative feature map,
wherein the encoder and the decoder are lightweight neural networks trained using feature map extraction knowledge and image reconstruction knowledge transmitted from a teacher neural network.

16. The imaging apparatus of claim 15, wherein the encoder is a model configured to extract quantitative features from the ultrasound data using multi-stage separable convolution.

17. The imaging apparatus of claim 15, wherein the encoder and the decoder become lightweight through neural network parameter quantization and are configured to extract quantitative features from the ultrasound data and reconstruct the quantitative features to output the quantitative image.

18. The imaging apparatus of claim 15, wherein the encoder and the decoder are
artificial intelligence model trained using an objective function including a first loss related to a difference from a correct image, a second loss related to a difference from a feature map extracted from the teacher neural network, and a third loss related to a difference from the quantitative image generated from the teacher neural network.

19. The imaging apparatus of claim 15, wherein the quantitative variables include at least one of attenuation coefficient (AC), speed of sound (SoS), effective scatterer concentration (ESC), and effective scatterer diameter (ESD).
